(19) United States
(12) Patent Application Publication
(10) Pub. No.: US 2010/0317620 A1
(43) Pub. Date: Dec. 16, 2010

(54) N-PHENYLIMIDAZO[1,2-a]PYRIDINE-2-CARBOXAMIDE COMPOUNDS, PREPARATION AND THERAPEUTIC USE THEREOF

(75) Inventor: Jean-Francois PEYRONEL, Palaiseau (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: sanofi-aventis, Paris (FR)

(21) Appl. No.: 12/828,384

(22) Filed: Jul. 1, 2010

Related U.S. Application Data
(63) Continuation of application No. PCT/FR2008/001839, filed on Dec. 31, 2008.

(30) Foreign Application Priority Data
Jan. 2, 2008 (FR) 0800008

(51) Int. Cl. A61K 31/695; C07D 471/04; A61K 31/437; C07F 7/10; A61P 25/00; A61P 25/14; A61P 25/28; A61P 29/00; A61P 35/00

(52) U.S. Cl. 514/63; 546/121; 514/300; 546/14

(57) ABSTRACT

Compounds of formula (I):

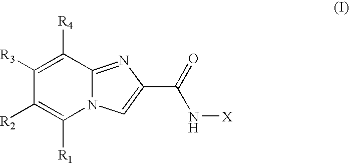

in which
X, R₁, R₂, R₃, and R₄ are as defined in the disclosure, or an acid addition salt thereof; and therapeutic uses thereof.

The present invention relates to imidazo[1,2-a]pyridine-2-carboxamide derivatives, to their preparation and to their therapeutic use in the treatment or prevention of diseases involving the Nurr-1 nuclear receptors, also known as NR4A2, NOT, TINUR, RNR-1 and HZF3.

One subject of the present invention is compounds of formula (I):

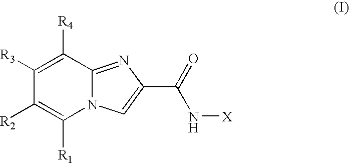

in which:

X represents a phenyl group optionally substituted with one or more groups chosen, independently of each other, from the following atoms or groups: halogen, (C₁-C₆)alkoxy, (C₁-C₆)alkyl, NRaRb, the alkyl and alkoxy groups possibly being substituted with one or more halogen atoms;

R₁ represents a hydrogen atom, a halogen atom, a group (C₁-C₆)alkoxy, a group (C₁-C₆)alkyl, an amino or a group NRcRd; the alkyl and alkoxy groups possibly being substituted with one or more halogen atoms, a hydroxyl or amino group, or a group (C₁-C₆)alkoxy;

R₂ represents one of the following groups:
a group NRcRd,
a group —N═CH—NRaRb,
nitro, hydroxyiminoalkyl, alkoxyiminoalkyl,
a group (C₁-C₆)alkylthio,
a group (C₁-C₆)alkylsulfinyl,
a group (C₁-C₆)alkylsulfonyl,
a group —SO₂—NR₅R₆,
a group (((C₁-C₆)alkyl)₃)silylethynyl;

R₃ represents a hydrogen atom, a group (C₁-C₆)alkyl, a group (C₁-C₆)alkoxy or a halogen atom,

R₄ represents a hydrogen atom, a group (C₁-C₄)alkyl, a group (C₁-C₄)alkoxy or a fluorine atom;

R₅ and R₆, which may be identical or different, represent a hydrogen atom or a group (C₁-C₆)alkyl;

Ra and Rb represent, independently of each other, a hydrogen atom or a group (C₁-C₆)alkyl or form, with the nitrogen atom that bears them, a 4- to 7-membered ring;

Rc represents a hydrogen atom and Rd represents a hydrogen atom or a group (C₁-C₆)alkyl, the said alkyl being optionally substituted with a group (C₁-C₆)alkoxy;

in the form of the base or of an acid-addition salt.

Document FR 2 638 161 discloses compounds derived from benzoyl-2-imidazo[1,2-a]pyridine, which are useful as medicaments.

The compounds of formula (I) may comprise one or more asymmetric carbon atoms. They may thus exist in the form of enantiomers or diastereoisomers. These enantiomers and diastereoisomers, and also mixtures thereof, including racemic mixtures, form part of the invention.

The compounds of formula (I) may exist in the form of bases or of acid-addition salts. Such addition salts form part of the invention.

These salts may be prepared with pharmaceutically acceptable acids, but the salts of other acids that are useful, for example, for purifying or isolating the compounds of formula (I) also form part of the invention.

The compounds of formula (I) may also exist in the form of hydrates or solvates, i.e. in the form of associations or combinations with one or more water molecules or with a solvent. Such hydrates and solvates also form part of the invention.

In the context of the present invention, the following definitions apply:
a halogen atom: a fluorine, a chlorine, a bromine or an iodine;
an alkyl group: a linear, branched or cyclic, saturated aliphatic group, optionally substituted with a linear, branched or cyclic, saturated alkyl group. Examples that may be mentioned include methyl, ethyl, propyl, isopropyl, butyl, isobutyl, tert-butyl, cyclopropyl, cyclobutyl, cyclopentyl, cyclohexyl, methylcyclopropyl, etc. groups;
   an alkenyl group: a linear or branched, mono- or polyunsaturated aliphatic group, comprising, for example, one or two ethylenic unsaturations;
   an alkoxy group: a radical —O-alkyl in which the alkyl group is as defined previously;
   an alkynyl group: a linear or branched, mono- or polyunsaturated aliphatic group, comprising, for example, one or two ethylynic unsaturations.

Various subgroups of compounds are defined hereinbelow, and also form part of the invention.

Among the compounds of general formula (I) that are subjects of the invention as defined previously, a first group of compounds is constituted by compounds for which at least one of the groups R₁ or R₂ is other than a hydrogen atom; the other groups being as defined previously.

Among the compounds of general formula (I) that are subjects of the invention as defined previously, a second group of compounds is constituted by compounds for which R₁ represents a hydrogen atom, a halogen atom, a group (C₁-C₆)alkyl or a group (C₁-C₆)alkoxy; the other groups being as defined previously.

Among the compounds of general formula (I) that are subjects of the invention as defined previously, a third group of compounds is constituted by compounds for which X represents a phenyl group; the other groups being as defined previously.

Among the compounds of general formula (I) that are subjects of the invention as defined previously, a fourth group of compounds is constituted by compounds for which R₃ and R₄ each represent a hydrogen atom; the other groups being as defined previously.

Among the compounds of general formula (I) that are subjects of the invention as defined previously, a fifth group of compounds is constituted by compounds for which R₂ represents one of the following groups:
a group NRcRd,
a group —N═CH—NRaRb,
nitro, hydroxyiminoalkyl, alkoxyiminoalkyl,
a group (C₁-C₆)alkylthio,
a group (C₁-C₆)alkylsulfinyl,
a group (C₁-C₆)alkylsulfonyl,
a group —SO₂—NR₅R₆,
a group (((C₁-C₆)alkyl)₃)silylethynyl;

R₅ and R₆, which may be identical or different, represent a hydrogen atom or a group (C₁-C₆)alkyl;

Ra and Rb represent, independently of each other, a hydrogen atom or a group (C₁-C₆)alkyl;

Rc represents a hydrogen atom and Rd represents a hydrogen atom or a group (C₁-C₆)alkyl, the said alkyl being optionally substituted with a group (C₁-C₆)alkoxy;

the other groups being as defined previously.

Among the compounds of general formula (I) that are subjects of the invention as defined previously, a sixth group of compounds is constituted by compounds for which R₂ represents one of the following groups:

NH₂, CH═NOH, NHiPr, nitro, CH═NOMe, NHMe, N═CHNMe₂, NHEt, NHCH₂CH₂OMe, SMe, SOMe, SO₂Me, SO₂NH₂, SO₂NHMe, SO₂NMe₂, C≡CSiMe₃;

the other groups being as defined previously.

Among the compounds of formula (I) that are subjects of the invention, a seventh group of compounds is constituted of compounds for which:

X represents a phenyl group;

R₁ represents a hydrogen atom, a chlorine atom or a methyl or ethoxy group; R₃ and R₄ represent a hydrogen atom;

R₂ represents a group NH₂, CH═NOH, NHiPr, nitro, CH═NOMe, NHMe, N═CHNMe₂, NHEt, NHCH₂CH₂OMe, SMe, SOMe, SO₂Me, SO₂NH₂, SO₂NHMe, SO₂NMe₂, C≡CSiMe₃;

in the form of the base or of an acid-addition salt.

Among the compounds of formula (I) that are subjects of the invention, mention may be made especially of the following compounds:
6-[(E)-(Hydroxyimino)methyl]-N-phenylimidazo[1,2-a]pyridine-2-carboxamide
6-(Isopropylamino)-N-phenylimidazo[1,2-a]pyridine-2-carboxamide and its hydrochloride (1:1)
6-Nitro-N-phenylimidazo[1,2-a]pyridine-2-carboxamide
6-Amino-N-phenylimidazo[1,2-a]pyridine-2-carboxamide
6-Amino-5-chloro-N-phenylimidazo[1,2-a]pyridine-2-carboxamide
6-Amino-5-ethoxy-N-phenylimidazo[1,2-a]pyridine-2-carboxamide
6-{[(1E)-(Dimethylamino)methylene]amino}-N-phenylimidazo[1,2-a]pyridine-2-carboxamide
6-[(E)-(Methoxyimino)methyl]-N-phenylimidazo[1,2-a]pyridine-2-carboxamide
6-(Methylamino)-N-phenylimidazo[1,2-a]pyridine-2-carboxamide
6-(Ethylamino)-N-phenylimidazo[1,2-a]pyridine-2-carboxamide
6-(2-Methoxyethylamino)-N-phenylimidazo[1,2-a]pyridine-2-carboxamide
5-Methyl-6-nitro-N-phenylimidazo[1,2-a]pyridine-2-carboxamide
6-Amino-5-methyl-N-phenylimidazo[1,2-a]pyridine-2-carboxamide
6-(Methylthio)-N-phenylimidazo[1,2-a]pyridine-2-carboxamide and its hydrobromide (1:1)
6-[(RS)-Methylsulfinyl]-N-phenylimidazo[1,2-a]pyridine-2-carboxamide and its hydrobromide (1:1)
6-(Methylsulfonyl)-N-phenylimidazo[1,2-a]pyridine-2-carboxamide and its hydrobromide (1:1)
6-(Aminosulfonyl)-N-phenylimidazo[1,2-a]pyridine-2-carboxamide and its hydrobromide (1:1)
6-[(Methylamino)sulfonyl]-N-phenylimidazo[1,2-a]pyridine-2-carboxamide and its hydrobromide (1:1)
6-[(Dimethylamino)sulfonyl]-N-phenylimidazo[1,2-a]pyridine-2-carboxamide
6-(Trimethylsilylethynyl)-N-phenylimidazo[1,2-a]pyridine-2-carboxamide and its hydrobromide (1:1)

The acid-addition salts of these compounds also form part of the invention.

In accordance with the invention, the compounds of general formula (I) may be prepared according to the process described in Scheme 1.

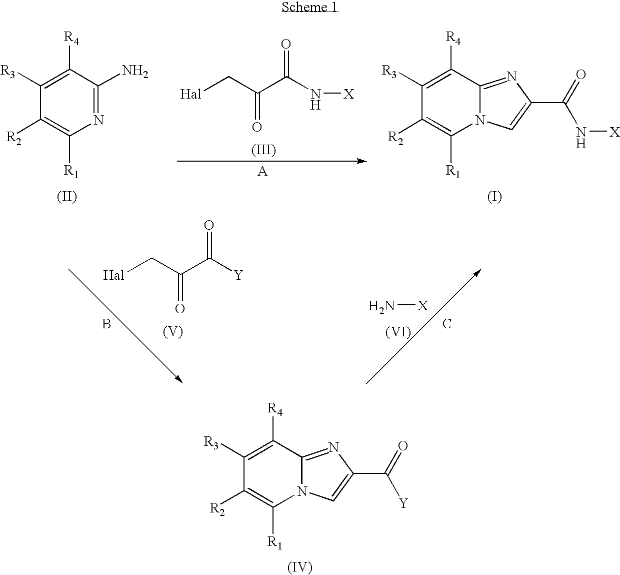

Route A consists in preparing the 2-aminopyridines of formula (II) according to the methods known to those skilled in the art and in forming the imidazo[1,2-a]pyridine ring by condensation with a 2-oxo-N-arylpropionamide derivative (III) in which Hal represents a chlorine, bromine or iodine atom and X is as defined previously, by analogy with the methods described by J-J. Bourguignon et al. in Aust. J. Chem., 50, 719 (1997) and by J. G. Lombardino in J. Org. Chem., 30, 2403 (1965), for example. The halo derivatives of the 2-oxo-N-aryl-propionamide (III) may be obtained according to the method described by R. Kluger et al. in J. Am. Chem. Soc., 106, 4017 (1984).

The second synthetic route B-C consists in coupling an imidazopyridine-2-carboxylic acid or a derivative thereof, of formula (IV), in which Y is hydroxyl, halogen or (C₁-C₆)alkoxy, with an arylamine X—NH₂ of formula (VI), in which X is as defined previously, according to methods known to those skilled in the art. Thus, the acid may be converted beforehand into a reactive derivative thereof such as an acid halide, anhydride, mixed anhydride or activated ester, and then reacted with the amine (VI) in the presence of a base such as diisopropylethylamine, triethylamine or pyridine, in an inert solvent such as THF, DMF or dichloromethane. The coupling may also be performed in the presence of a coupling agent such as CDI, EDCI, HATU or HBTU under the same conditions without isolating the reaction intermediate. Alternatively, the amine (VI) may be reacted with an ester of the acid of formula (IV) in the presence of a catalyst such as trimethylaluminium, according to the method of Weinreb, S. et al. (Tet. Lett. (1977), 18, 4171), or zirconium tert-butoxide.

The imidazopyridine-2-carboxylic acids and the derivatives thereof of formula (IV) may be obtained by condensing the appropriate 2-aminopyridines with a 3-halo-2-oxopropionic acid ester according to the method described by J. G. Lombardino in J. Org. Chem., 30(7), 2403 (1965), followed by deprotecting the ester to the acid and, where appropriate, converting the acid into a derivative thereof.

The products of formula (I) and the precursors thereof of formula (II) or (IV) may be subjected, if desired and if necessary, in order to obtain products of formula (I) or to be converted into other products of formula (I), to one or more of the following transformation reactions, in any order:

a) a reaction for the esterification or amidation of an acid function,

b) a reaction for the amidation of an amine function,

c) a reaction for the hydrolysis of an ester function to an acid function,

d) a reaction for the transformation of a hydroxyl function into an alkoxy function,

e) a reaction for the oxidation of an alcohol function to an aldehyde or ketone function,

f) a reaction for the conversion of aldehyde or ketone functions into an oxime derivative,

g) a reaction for the transformation of a nitrile radical into an aldehyde function,

h) a reaction for the transformation of a nitrile radical into a ketone function,

i) a reaction for the oxidation of an alkenyl group into an aldehyde or ketone function,

j) a reaction for the reduction of a nitro group to a primary amino group,

k) a reaction for the conversion of a primary or secondary amino group into a secondary or tertiary amino group via reductive amination or alkylation,

l) a reaction for the conversion of a primary amino group into an amidine group,

m) a reaction for the oxidation of an alkylthioether function to an alkyl sulfoxide or alkyl sulfone function,

n) a reaction for the oxidation of an alkyl sulfoxide function to an alkyl sulfone function,

o) a reaction for the protection of reactive functions,

p) a reaction for the removal of the protecting groups that may be borne by the protected reactive functions,

q) a salification reaction with a mineral or organic acid or with a base, to obtain the corresponding salt,

r) a reaction for the resolution of racemic forms into enantiomers,

the said products of formula (I) thus obtained being, where appropriate, in any possible isomeric form as racemic mixtures, enantiomers, diastereoisomers or tautomers.

In Scheme 1, the starting compounds and the reagents, when their mode of preparation is not described, are commercially available or are described in the literature, or else may be prepared according to methods that are described therein or that are known to those skilled in the art.

The examples that follow describe the preparation of certain compounds in accordance with the invention. These examples are not limiting, but serve merely to illustrate the present invention. The numbers of the illustrated compounds refer to those given in the table hereinbelow, which illustrates the chemical structures and physical properties of a number of compounds according to the invention.

### EXAMPLE 1

### 6-[(E)-(Hydroxyimino)methyl]-N-phenylimidazo[1,2-a]pyridine-2-carboxamide

A solution of 123 mg of 6-formyl-N-phenylimidazo[1,2-a]pyridine-2-carboxamide and 48 mg of hydroxylamine hydrochloride in 6.5 mL of pyridine is stirred for 2 hours at 20° C. and then concentrated to dryness under reduced pressure. The solid is washed with water and then with diethyl ether and dissolved in a mixture of 75 mL of methanol and 75 mL of dichloromethane containing 2 mL of pyridine. The solution is evaporated in the presence of 1.5 g of silica. After chromatography on a column of silica, eluting with a mixture of cyclohexane and ethyl acetate, the fractions containing the expected product are combined and evaporated to dryness under reduced pressure. The solid is triturated with ethyl acetate, filtered off and washed with ethyl acetate and then with diethyl ether and dried to give 48 mg of 6-[(E)-(hydroxyimino)methyl]-N-phenylimidazo[1,2-a]pyridine-2-carboxamide in the form of a white solid.

### EXAMPLE 2

### 6-(Isopropylamino)-N-phenylimidazo[1,2-a]pyridine-2-carboxamide and its hydrochloride (1:1).

### 2.1: 5-Isopropylaminopyridine-2-amine

After having slowly added 1 g of stannous chloride to a suspension of 362 mg of 5-isopropylamino-6-nitropyridine (WO 2006/040 520) in 10 mL of ethanol, the reaction mixture is refluxed for 30 minutes and then concentrated to dryness. The residue is taken up in ammoniacal methanol, the mixture is filtered and the filtrate is concentrated to dryness. The residue is chromatographed on a silica cartridge, eluting with a gradient of dichloromethane and ethyl acetate (from 0/100 to 100/0) and then with a 90/10 mixture of dichloromethane and 7N ammoniacal methanol. The fractions containing the expected product are combined and concentrated to dryness to give 143 mg of 5-isopropylaminopyridine-2-amine in the form of a violet-coloured oil.

¹H NMR spectrum (DMSO-d6, δ in ppm): 1.08 (d, J=6.5 Hz, 6H), 3.38 (m, 1H), 4.58 (broad m, 1H), 5.18 (broad m, 2H), 6.38 (d, J=8.5 Hz, 1H), 6.89 (dd, J=3.0 and 8.5 Hz, 1H), 7.33 (d, J=3.0 Hz, 1H).

Mass spectrum (LC-MS-DAD-ELSD): m/z 152 [M+H]⁺.

### 2.2: 6-(Isopropylamino)-N-phenylimidazo[1,2-c]pyridine-2-carboxamide and its hydrochloride (1:1)

To a solution of 143 mg of 5-isopropylaminopyridine-2-amine in 10 mL of 1,2-dimethoxyethane and 1 mL of ethanol are added 172 mg of 3-bromo-2-oxo-N-phenylpropionamide. The reaction mixture is stirred for 90 hours at 25° C. and then refluxed for 2 hours and concentrated to dryness under reduced pressure. The residue is taken up in a mixture of dichloromethane and saturated sodium bicarbonate solution. The organic phase is dried over magnesium sulfate and concentrated to dryness under reduced pressure. The residue is chromatographed on a silica cartridge, eluting with a mixture of dichloromethane and ethyl acetate (80/20). The fractions containing the expected product are combined and concentrated to dryness to give 136 mg of 6-(isopropylamino)-N-phenylimidazo[1,2-a]pyridine-2-carboxamide in the form of a brown solid.

This product is taken up in dioxane containing a small amount of methanol and treated with 116 μL of a 4N solution of hydrogen chloride in dioxane. After stirring for 1 hour at 20° C., the precipitate is filtered off by suction, washed with dioxane and dried to give 136 mg of 6-(isopropylamino)-N-phenylimidazo[1,2-c]pyridine-2-carboxamide hydrochloride (1:1) in the form of a pale pink solid.

### EXAMPLE 3

### 6-Nitro-N-phenylimidazo[1,2-a]pyridine-2-carboxamide

To a solution of 638 μL of aniline in 78 mL of toluene, cooled to 0° C., are added dropwise 4.68 mL of a 2M solution of trimethylaluminium in toluene, followed, at 20° C., by addition of 800 mg of ethyl 6-nitroimidazo[1,2-a]pyridine-2-carboxylate (Heterocycles 38(7), 1527 (1994)). The reaction mixture is stirred for 2 hours at room temperature and then cooled to 4° C. and treated slowly with 40 mL of saturated ammonium chloride solution, concentrated under reduced pressure and rediluted with 150 mL of water and 400 mL of ethyl acetate. The organic phase is dried over magnesium sulfate, filtered through Celite and evaporated to dryness under reduced pressure. The residue is triturated with dichloromethane, filtered off by suction and dried to give 247 mg of 6-nitro-N-phenylimidazo[1,2-a]pyridine-2-carboxamide) in the form of a green-grey solid.

### EXAMPLE 4

### 6-Amino-N-phenylimidazo[1,2-a]pyridine-2-carboxamide

To a suspension of 190 mg of 6-nitro-N-phenylimidazo[1,2-a]pyridine-2-carboxamide in 3 mL of ethanol are added 562 mg of stannous chloride and the mixture is refluxed for 30 minutes. The reaction mixture is concentrated to dryness and the residue is taken up in a solution of ammonia in methanol (7N). The suspension is filtered and the filtrate is evaporated to dryness under reduced pressure. The residue is chromatographed on a silica cartridge, eluting with a 50/50 mixture of dichloromethane and ethyl acetate. The fractions containing the expected product are combined and evaporated to dryness under reduced pressure to give 30 mg of 6-amino-N-phenylimidazo[1,2-a]pyridine-2-carboxamide in the form of a green solid.

Chromatography enables the products of Examples 5 and 6 to be isolated.

### EXAMPLE 5

### 6-Amino-5-chloro-N-phenylimidazo[1,2-a]pyridine-2-carboxamide

10 mg of 6-amino-5-chloro-N-phenylimidazo[1,2-a]pyridine-2-carboxamide are obtained as by-product of the preparation of the compound of Example 4. The product is isolated by chromatography on silica, in the form of a green solid.

### EXAMPLE 6

### 6-Amino-5-ethoxy-N-phenylimidazo[1,2-a]pyridine-2-carboxamide

16 mg of 6-amino-5-ethoxy-N-phenylimidazo[1,2-a]pyridine-2-carboxamide are obtained as by-product of the preparation of the compound of Example 4. The product is isolated by chromatography on silica, in the form of a green solid.

### EXAMPLE 7

### 6-{[(1E)-(Dimethylamino)methylene]amino}-N-phenylimidazo[1,2-a]pyridine-2-carboxamide

### 7.1: Ethyl 6-{[(1E)-(dimethylamino)methylene]amino}-5-methylimidazo[1,2-a]pyridine-2-carboxylate

200 mg of ethyl 6-aminoimidazo[1,2-a]pyridine-2-carboxylate (Heterocycles 38(7), 1527 (1994)) are treated with 2 mL of N,N-dimethylformamide dimethyl acetal. The reaction mixture is refluxed for 2 hours and then cooled and diluted with pentane. The precipitate is filtered off by suction, washed and dried to give 180 mg of ethyl 6-{[(1E)-(dimethylamino)methylene]amino}-5-methylimidazo[1,2-a]pyridine-2-carboxylate in the form of an ochre-coloured solid.

¹H NMR spectrum (DMSO-d6, δ in ppm): 1.31 (t, J=3H), 2.91 (broad s, 3H), 3.02 (broad s, 3H), 4.29 (q, J=7.0 Hz, 2H), 7.21 (dd, J=1.5 and 9.5 Hz, 1H), 7.47 (d, J=9.5 Hz, 1H), 7.84 (s, 1H), 8.05 (d, J=1.5 Hz, 1H), 8.35 (s, 1H).

Mass spectrum (LC-MS-DAD-ELSD): m/z 261 [M+H]⁺, m/z 233 [MH-C₂H₅]⁺

### 7.2: 6-{[(1E)-(Dimethylamino)methylene]amino}-N-phenylimidazo[1,2-a]pyridine-2-carboxamide

By working in a manner similar to that of Example 2 (step 2.2), starting with 112 mg of ethyl 6-{[(1E)-(dimethylamino)methylene]amino}-5-methylimidazo[1,2-a]pyridine-2-carboxylate, 79 mg of 6-{[(1E)-(dimethylamino)methylene]amino}-N-phenylimidazo[1,2-a]pyridine-2-carboxamide are obtained in the form of a beige-coloured solid.

The intermediates described below are useful for preparing the compounds of the present invention.

### 5-Ethylaminopyridine-2-amine

5-Ethylaminopyridine-2-amine is prepared in the same manner as 5-isopropylamino-pyridine-2-amine (Example 2.1) starting with 5-ethylamino-6-nitropyridine (PCT Int. Appl. WO 2006/040 520).

### 5-(2-Methoxyethylamino)pyridine-2-amine

370 mg of 5-(2-methoxyethylamino)-2-nitropyridine (WO 2006/040 526) are added slowly to a suspension of 597 mg of tin in 6 mL of 48% hydrobromic acid cooled to −5° C. The reaction mixture is stirred for 2 hours at −5° C. 6 mL of 25% aqueous ammonia are added slowly and the mixture is extracted with dichloromethane. The organic phase is separated out by settling, dried over magnesium sulfate and concentrated to dryness under reduced pressure to give the crude 5-(2-methoxyethylamino)pyridine-2-amine in the form of a brown oil, which is used in the remainder of the synthesis without further purification.

Mass spectrum (LC/MS): m/z 168: [M+H]⁺.

The tables that follow illustrate the chemical structures (Table 1) and the spectroscopic characteristics (Table 2) of a number of examples of compounds according to the invention.

In these tables, “HCl” means hydrochloride; “HBr” means hydrobromide; “-” means that the compound is in the form of the base; “Me” means a methyl group, “Et” means an ethyl group, “iPr” means an isopropyl group and “OMe” means a methoxy group.

TABLE 1
(I)

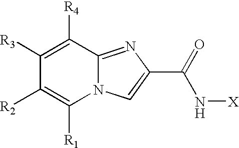

| Ex | R₁ | R₂ | R₃ | R₄ | X | salt |
|---|---|---|---|---|---|---|
| 1 | H | CH═NOH | H | H | Ph | — |
| 2 | H | NHiPr | H | H | Ph | HCl |
| 3 | H | NO₂ | H | H | Ph | — |
| 4 | H | NH₂ | H | H | Ph | — |
| 5 | Cl | NH₂ | H | H | Ph | — |
| 6 | OEt | NH₂ | H | H | Ph | — |
| 7 | H | N═CHNMe₂ | H | H | Ph | — |
| 8 | H | CH═NOMe | H | H | Ph | — |
| 9 | H | NHMe | H | H | Ph | — |
| 10 | H | NHEt | H | H | Ph | — |
| 11 | H | NHCH₂CH₂OMe | H | H | Ph | — |
| 12 | Me | NO₂ | H | H | Ph | — |
| 13 | Me | NH₂ | H | H | Ph | — |
| 14 | H | SMe | H | H | Ph | HBr |
| 15 | H | SOMe | H | H | Ph | HBr |
| 16 | H | SO₂Me | H | H | Ph | HBr |
| 17 | H | SO₂NH₂ | H | H | Ph | HBr |
| 18 | H | SO₂NHMe | H | H | Ph | HBr |
| 19 | H | SO₂NMe₂ | H | H | Ph | — |
| 20 | H | C≡CSiMe₃ | H | H | Ph | HBr |

TABLE 2

| Ex | Characterizations |
|---|---|
| 1 | ¹H NMR spectrum (DMSO-d6, δ in ppm): 7.09 (t J = 7.5 Hz, 1H), 7.34 (t, J = 7.5 Hz, 2H), |
| | from 7.62 to 7.72 (m, 2H), 7.89 (d, J = 7.5 Hz, 2H), 8.20 (s, 1H), 8.55 (s, 1H), 8.79 (broad s, |
| | 1H), 10.2 (s, 1H), 11.45 (broad s, 1H). |
| | Mass spectrum (LC-MS-DAD-ELSD): m/z 281 [M + H]⁺. |
| 2 | ¹H NMR spectrum (DMSO-d6, δ in ppm): 1.20 (d, J = 6.5 Hz, 6H), 3.47 (m, 1H), 7.14 (t, J = 8.0 Hz, |
| | 1H), 7.35 (partially masked m, 1H), 7.39 (t, J = 8.0 Hz, 2H), 7.58 (d, J = 9.5 Hz, |
| | 1H), 7.81 (d, J = 8.0 Hz, 2H), 7.92 (broad s, 1H), 8.59 (broad s, 1H), 10.6 (broad m, 1H). |
| | Mass spectrum (LC-MS-DAD-ELSD): m/z 295 [M + H]⁺. |
| 3 | ¹H NMR spectrum (DMSO-d6, δ in ppm): 7.11 (t, J = 8.0 Hz, 1H), 7.36 (t, J = 8.0 Hz, 2H), |
| | 7.82 (d, J = 9.5 Hz, 1H), 7.89 (d, J = 8.0 Hz, 2H), 8.08 (dd, J = 2.0 and 9.5 Hz, 1H), 8.74 (s, |
| | 1H), 9.96 (d, J = 2.0 Hz, 1H), 10.4 (broad s, 1H). |
| | Mass spectrum (LC-MS-DAD-ELSD): m/z 283 [M + H]⁺. |
| 4 | ¹H NMR spectrum (DMSO-d6, δ in ppm): 5.10 (broad s, 2H), 6.99 (dd, J = 1.5 and 9.5 Hz, |
| | 1H), 7.06 (t, J = 7.5 Hz, 1H), 7.31 (t, J = 7.5 Hz, 2H), 7.41 (d, J = 9.5 Hz, 1H), 7.71 (d, J = 1.5 Hz, |
| | 1H), 7.87 (d, J = 7.5 Hz, 2H), 8.29 (s, 1H), 10.05 (s, 1H). |
| | Mass spectrum (LC-MS-DAD-ELSD): m/z 252 [M]⁺. |
| 5 | ¹H NMR spectrum (DMSO-d6, δ in ppm): 5.50 (s, 2H), 7.09 (t, J = 7.5 Hz, 1H), 7.20 (d, J = 9.5 Hz, |
| | 1H), 7.32 (t, J = 7.5 Hz, 2H), 7.53 (d, J = 9.5 Hz, 1H), 7.88 (d, J = 7.5 Hz, 2H), |
| | 8.19 (s, 1H), 10.15 (s, 1H). |
| | Mass spectrum (LC-MS-DAD-ELSD): m/z 287 [M + H]⁺, presence of 1 Cl. |
| 6 | ¹H NMR spectrum (DMSO-d6, δ in ppm): 1.43 (t, J = 7.0 Hz, 3H), 4.19 (q, J = 7.0 Hz, 2H), |
| | 4.82 (broad s, 2H), 7.07 (t, J = 7.5 Hz, 1H), 7.15 (d, J = 9.0 Hz, 1H), 7.31 (d, J = 9.0 Hz, |
| | 1H), 7.33 (t, J = 7.5 Hz, 2H), 7.89 (d, J = 7.5 Hz, 2H), 8.12 (s, 1H), 10.1 (s, 1H). |
| | Mass spectrum (LC-MS-DAD-ELSD): m/z 297 [M + H]⁺. |
| 7 | ¹H NMR spectrum (DMSO-d6, δ in ppm): 2.93 (broad s, 3H), 3.03 (broad s, 3H), 7.09 (t, J = 7.5 Hz, |
| | 1H), 7.24 (dd, J = 1.5 and 9.5 Hz, 1H), 7.33 (t, J = 7.5 Hz, 2H), 7.51 (d, J = 9.5 Hz, |
| | 1H), 7.87 (s, 1H), 7.89 (d, J = 7.5 Hz, 2H), 8.11 (broad s, 1H), 8.34 (s, 1H), 10.1 (s, 1H). |
| | Mass spectrum (LC-MS-DAD-ELSD): m/z 308 [M + H]⁺. |
| 8 | ¹H NMR spectrum (DMSO-d6, δ in ppm): 3.92 (s, 3H), 7.10 (t J = 7.5 Hz, 1H), 7.34 (t, J = 7.5 Hz, |
| | 2H), from 7.62 to 7.72 (m, 2H), 7.89 (d, J = 7.5 Hz, 2H), 8.30 (s, 1H), 8.59 (s, 1H), |
| | 8.83 (broad s, 1H), 10.25 (broad s, 1H). |
| | Mass spectrum (LC-MS-DAD-ELSD): m/z 295 [M + H]⁺. |
| 9 | ¹H NMR spectrum (DMSO-d6, δ in ppm): 2.68 (d, J = 5.5 Hz, 3H), 5.72 (q, J = 5.5 Hz, 1H), |
| | 6.99 (dd, J = 1.5 and 9.5 Hz, 1H), 7.08 (t, J = 7.5 Hz, 1H), 7.32 (t, J = 7.5 Hz, 2H), 7.41 (d, J = 9.5 Hz, |
| | 1H), 7.58 (d, J = 1.5 Hz, 1H), 7.87 (broad d, J = 7.5 Hz, 2H), 8.29 (s, 1H), 10.0 (s, |
| | 1H). |
| | Mass spectrum (LC-MS-DAD-ELSD): m/z 267 [M + H]⁺. |
| 10 | ¹H NMR spectrum (DMSO-d6, “ in ppm): 1.22 (t, J = 7.5 Hz, 3H), 2.98 (m, 2H), 5.62 (t, J = 5.5 Hz, |
| | 1H), 7.00 (dd, J = 1.5 and 9.5 Hz, 1H), 7.07 (broad t, J = 7.5 Hz, 1H), 7.31 (t, J = 7.5 Hz, |
| | 2H), 7.42 (d, J = 9.5 Hz, 1H), 7.61 (d, J = 1.5 Hz, 1H), 7.87 (d, J = 7.5 Hz, 2H), 8.27 (s, |
| | 1H), 10.0 (s, 1H). |
| | Mass spectrum (LC-MS-DAD-ELSD): m/z 281 [M + H]⁺. |
| 11 | ¹H NMR spectrum (DMSO-d6, δ in ppm): 3.12 (q, J = 6.0 Hz, 2H), 3.30 (masked s, 3H), |
| | 3.58 (t, J = 6.0 Hz, 2H), 5.70 (t, J = 6.0 Hz, 1H), 7.07 (m, 2H), 7.31 (t, J = 7.5 H, 2H), |
| | 7.42 (d, J = 9.5 Hz, 1H), 7.69 (d, J = 1.5 Hz, 1H), 7.88 (d, J = 7.5 Hz, 2H), 8.25 (s, 1H), 10.0 (s, |
| | 1H). |
| | Mass spectrum (LC-MS-DAD-ELSD): m/z 311 [M + H]⁺. |
| 12 | ¹H NMR spectrum (DMSO-d6, δ in ppm): 3.02 (s, 3H), 7.11 (t, J = 7.5 Hz, 1H), 7.37 (t, J = 7.5 Hz, |
| | 2H), 7.73 (d, J = 9.5 Hz, 1H), 7.90 (d, J = 7.5 Hz, 2H), 7.97 (d, J = 9.5 Hz, 1H), |
| | 8.81 (s, 1H), 10.4 (s, 1H). |
| | Mass spectrum (LC-MS-DAD-ELSD): m/z 295 [M − H]⁻, m/z 297 [M + H]⁺. |
| 13 | ¹H NMR spectrum (DMSO-d6, δ in ppm): 2.41 (s, 3H), 4.93 (broad s, 2H), 7.08 (m, 2H), |
| | 7.32 (t, J = 7.5 Hz, 2H), 7.38 (d, J = 9.5 Hz, 1H), 7.89 (d, J = 7.5 Hz, 2H), 8.13 (s, 1H), |
| | 10.1 (s, 1H). |
| | Mass spectrum (LC-MS-DAD-ELSD): m/z 267 [M + H]⁺, m/z = 192 [MH − Ph]⁺, m/z = 174 [MH − NHPh]⁺. |
| 14 | ¹H NMR spectrum (DMSO-d6, δ in ppm): 2.58 (s, 3H), 7.12 (t, J = 8.0 Hz, 1H), 7.38 (t, J = 8.0 Hz, |
| | 2H), 7.58 (broad d, J = 9.5 Hz, 1H), 7.69 (d, J = 9.5 Hz, 1H), 7.85 (d, J = 8.0 Hz, |
| | 2H), 8.59 (s, 1H), 8.69 (broad s, 1H), 10.5 (s, 1H). |
| | Mass spectrum (LC-MS-DAD-ELSD): m/z 283 [M]⁺. |
| 15 | ¹H NMR spectrum (DMSO-d6, δ in ppm): 2.90 (s, 3H), 7.11 (t, J = 7.5 Hz, 1H), 7.36 (t, J = 7.5 Hz, |
| | 2H), 7.69 (broad d, J = 9.5 Hz, 1H), 7.84 (d, J = 9.5 Hz, 1H), 7.89 (d, J = 7.5 Hz, |
| | 2H), 8.70 (s, 1H), 9.04 (broad s, 1H), 10.35 (s, 1H). |
| | Mass spectrum (LC-MS-DAD-ELSD): m/z 298 [M − H]⁻, m/z 300 [M + H]⁺. |
| 16 | ¹H NMR spectrum (DMSO-d6, δ in ppm): 3.36 (s, 3H), 7.11 (t, J = 7.5 Hz, 1H), 7.35 (t, J = 7.5 Hz, |
| | 2H), 7.78 (broad d, J = 9.5 Hz, 1H), 7.89 (m, 3H), 8.75 (s, 1H), 9.39 (broad s, 1H), |
| | 10.4 (s, 1H). |
| | Mass spectrum (LC-MS-DAD-ELSD): m/z 314 [M − H]⁻, m/z 316 [M + H]⁺. |
| 17 | ¹H NMR spectrum (DMSO-d6, δ in ppm): 7.11 (t, J = 7.5 Hz, 1H), 7.35 (t, J = 7.5 Hz, 2H), |
| | 7.67 (broad s, 2H), 7.69 (broad d, J = 9.5 Hz, 1H), 7.86 (d, J = 9.5 Hz, 1H), 7.89 (d, J = 7.5 Hz, |
| | 2H), 8.73 (s, 1H), 9.26 (broad s, 1H), 10.35 (s, 1H). |
| | Mass spectrum (LC-MS-DAD-ELSD): m/z 315 [M − H]⁻, m/z 317 [M + H]⁺. |
| 18 | ¹H NMR spectrum (DMSO-d6, δ in ppm): 2.50 (masked m, 3H), 7.11 (t, J = 7.5 Hz, 1H), |
| | 7.36 (t, J = 7.5 Hz, 2H), 7.61 (broad d, J = 9.5 Hz, 1H), 7.80 (broad q, J = 5.5 Hz, 1H), |
| | 7.88 (m, 3H), 8.71 (s, 1H), 9.27 (broad s, 1H), 10.4 (s, 1H). |
| | Mass spectrum (LC-MS-DAD-ELSD): m/z 329 [M − H]⁻, m/z 331 [M + H]⁺. |
| 19 | ¹H NMR spectrum (DMSO-d6, δ in ppm): 2.74 (s, 6H), 7.11 (t, J = 7.5 Hz, 1H), 7.36 (t, J = 7.5 Hz, |
| | 2H), 7.58 (dd, J = 1.5 and 9.5 Hz, 1H), 7.82 (d, J = 9.5 Hz, 1H), 7.90 (d, J = 7.5 Hz, |
| | 2H), 8.69 (s, 1H), 9.30 (broad s, 1H), 10.35 (s, 1H). |
| | Mass spectrum (LC-MS-DAD-ELSD): m/z 343 [M − H]⁻, m/z 345 [M + H]⁺. |
| 20 | ¹H NMR spectrum (DMSO-d6, δ in ppm): 0.27 (s, 9H), 7.12 (t, J = 7.7 Hz, 1H), 7.36 (t, J = 7.7 Hz, |
| | 2H), 7.47 (dd, J = 9.3, 1.5 Hz, 1H), 7.68 (d, J = 9.3 Hz, 1H), 7.87 (broad d, J = 7.8 Hz, 2H), |
| | 8.54 (s, 1H), 9.02 (broad s, 1H), 10.41 (s, 1 H). |
| | Mass spectrum (LC-MS-DAD-ELSD): m/z 334 [M + H]⁺. |

The compounds according to the invention underwent pharmacological tests to determine their modulatory effect on NOT.

Evaluation of the In Vitro Activity on N2A Cells

The activity of the compounds according to the invention was evaluated on a cell line (N2A) endogenously expressing the murine Nurr1 receptor and stably transfected with the NOT binding response element (NBRE) coupled to the luciferase reporter gene. The EC₅₀ values are between 0.01 and 1000 nM. The tests were performed according to the procedure described hereinbelow.

The cell line Neuro-2A is obtained from a standard commercial source (ATCC). The clone Neuro-2A was obtained from a spontaneous tumour originating from a strain of albino mice A by R. J Klebe et al. This line Neuro-2A is then stably transfected with 8NBRE-luciferase. The N2A-8NBRE cells are cultured to the point of confluence in 75 cm² culture flasks containing DMEM supplemented with 10% foetal calf serum, 4.5 g/L of glucose and 0.4 mg/ml of geneticin. After culturing for one week, the cells are recovered with 0.25% trypsin for 30 seconds and then resuspended in DMEM without phenol red, containing 4.5 g/L of glucose and 10% Hyclone defatted serum, and placed in white, transparent-based 96-well plates. The cells are deposited at a rate of 60 000 per well in 75 μL for 24 hours before adding the products. The products are applied in 25 μL and incubated for a further 24 hours. On the day of measurement, an equivalent volume (100 μL) of Steadylite is added to each well, and the wells are then left for 30 minutes to obtain complete lysis of the cells and maximum production of the signal. The plates are then measured in a microplate luminescence counter, after having been sealed with an adhesive film. The products are prepared in the form of a 10⁻² M stock solution, and then diluted in 100% of DMSO. Each concentration of product is prediluted in culture medium before incubation with the cells thus containing 0.625% final of DMSO.

For example, compounds 2, 14 and 16 gave EC₅₀ values, respectively, of 1.6 nM, 2 nM and 16 nM.

It is thus seen that the compounds according to the invention have a modulatory effect on NOT.

The compounds according to the invention may thus be used for the preparation of medicaments for their therapeutic application in the treatment or prevention of diseases involving the NOT receptors.

Thus, according to another of its aspects, a subject of the invention is medicaments comprising a compound of formula (I), or an addition salt thereof with a pharmaceutically acceptable acid.

These medicaments find their therapeutic use especially in the treatment and prevention of neurodegenerative diseases, for instance Parkinson's disease, Alzheimer's disease, tauopathies (e.g. progressive supranuclear palsy, frontotemporal dementia, corticobasal degeneration, Pick's disease); cerebral trauma, for instance ischaemia and cranial trauma and epilepsy; psychiatric diseases, for instance schizophrenia, depression, substance dependency and attention-deficit hyperactivity disorder; inflammatory diseases of the central nervous system, for instance multiple sclerosis, encephalitis, myelitis and encephalomyelitis and other inflammatory diseases, for instance vascular pathologies, atherosclerosis, joint inflammations, arthrosis, rheumatoid arthritis; osteoarthritis, Crohn's disease, ulcerative colitis; allergic inflammatory diseases such as asthma, autoimmune diseases, for instance type 1 diabetes, lupus, scleroderma, Guillain-Barrésyndrome, Addison's disease and other immune-mediated diseases; osteoporosis; cancers.

Thus, the present invention is directed towards a compound chosen from the compounds of formula (I) defined previously, for the treatment or prevention of one of the above-mentioned diseases.

According to one particular embodiment, these medicaments find their use in the treatment or prevention of one of the abovementioned diseases, with the exception of inflammatory diseases.

According to another of its aspects, the present invention relates to the use of a compound chosen from the compounds of formula (I) as defined previously, for the preparation of a medicament for treating or preventing one of the diseases mentioned hereinabove.

These compounds may also be used as a treatment combined with grafts and/or transplantations of stem cells.

According to another of its aspects, the present invention relates to pharmaceutical compositions comprising, as active principle, a compound according to the invention. These pharmaceutical compositions contain an effective dose of at least one compound according to the invention, or a pharmaceutically acceptable salt of the said compound, and also at least one pharmaceutically acceptable excipient.

The said excipients are chosen, according to the pharmaceutical form and the desired mode of administration, from the usual excipients known to those skilled in the art.

In the pharmaceutical compositions of the present invention for oral, sublingual, subcutaneous, intramuscular, intravenous, topical, local, intratracheal, intranasal, transdermal or rectal administration, the active principle of formula (I) above, or the salt thereof, may be administered in unit administration form, as a mixture with standard pharmaceutical excipients, to man and animals for the prophylaxis or treatment of the above complaints or diseases.

The appropriate unit forms of administration include oral forms such as tablets, soft or hard gel capsules, powders, granules and oral solutions or suspensions, sublingual, buccal, intratracheal, intraocular, intranasal or inhalation administration forms, topical, transdermal, subcutaneous, intramuscular or intravenous administration forms, rectal administration forms and implants. For topical application, the compounds according to the invention may be used in creams, gels, ointments or lotions.

By way of example, a unit administration form of a compound according to the invention in tablet form may comprise the following components:

| | | |
|---|---|---|
| Compound according to the invention | 50.0 | mg |
| Mannitol | 223.75 | mg |
| Croscarmellose sodium | 6.0 | mg |
| Corn starch | 15.0 | mg |
| Hydroxypropylmethylcellulose | 2.25 | mg |
| Magnesium stearate | 3.0 | mg |

There may be particular cases in which higher or lower dosages are appropriate; such dosages are not outside the context of the invention. According to the usual practice, the dosage that is appropriate for each patient is determined by the doctor according to the mode of administration and the weight and response of the said patient.

According to another of its aspects, the present invention also relates to a method for treating the pathologies indicated above, which comprises the administration, to a patient, of an effective dose of a compound according to the invention, or a pharmaceutically acceptable salt thereof.

It is understood that all the subjects of the invention defined above, especially the medicament, pharmaceutical composition and treatment method, also apply more particularly to the subgroups of compounds previously defined.

What is claimed is:

1. A compound of formula (I): 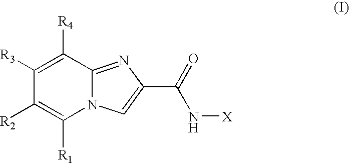
wherein:
X represents a phenyl group optionally substituted with one or more groups chosen, independently of each other, from the following atoms or groups: halogen, (C₁-C₆)alkoxy, (C₁-C₆)alkyl, and NRaRb, the alkyl and alkoxy groups possibly being substituted with one or more halogen atoms;
R₁ represents a hydrogen atom, a halogen atom, a group (C₁-C₆)alkoxy, a group (C₁-C₆)alkyl, an amino or a group NRcRd; the alkyl and alkoxy groups possibly being substituted with one or more halogen atoms, a hydroxyl or amino group, or a group (C₁-C₆)alkoxy;
R₂ represents one of the following groups:
a group NRcRd,
a group —N═CH—NRaRb,
nitro, hydroxyiminoalkyl, alkoxyiminoalkyl,
a group (C₁-C₆)alkylthio,
a group (C₁-C₆)alkylsulfinyl,
a group (C₁-C₆)alkylsulfonyl,
a group —SO₂—NR₅R₆, or
a group (((C₁-C₆)alkyl)₃)silylethynyl;
R₃ represents a hydrogen atom, a group (C₁-C₆)alkyl, a group (C₁-C₆)alkoxy or a halogen atom;
R₄ represents a hydrogen atom, a group (C₁-C₄)alkyl, a group (C₁-C₄)alkoxy or a fluorine atom;
R₅ and R₆, which may be identical or different, represent a hydrogen atom or a group (C₁-C₆)alkyl;
Ra and Rb represent, independently of each other, a hydrogen atom or a group (C₁-C₆)alkyl or form, with the nitrogen atom that bears them, a 4- to 7-membered ring; and
Rc represents a hydrogen atom and Rd represents a hydrogen atom or a group (C₁-C₆)alkyl, the said alkyl being optionally substituted with a group (C₁-C₆)alkoxy;
or an acid addition salt thereof.

2. The compound of formula (I) according to claim 1, wherein one of the groups R₁ or R₂ is other than a hydrogen atom; or an acid addition salt thereof.

3. The compound of formula (I) according to claim 1, wherein R₁ represents a hydrogen atom, a halogen atom or a group (C₁-C₆)alkoxy; or an acid addition salt thereof.

4. The compound of formula (I) according to claim 1, wherein R₃ and R₄ each represent a hydrogen atom; or an acid addition salt thereof.

5. The compound of formula (I) according to claim 1, wherein R₂ represents one of the following groups:
a group NRcRd,
a group —N═CH—NRaRb,
nitro, hydroxyiminoalkyl, alkoxyiminoalkyl,
a group (C₁-C₆)alkylthio,
a group (C₁-C₆)alkylsulfinyl,
a group (C₁-C₆)alkylsulfonyl,
a group —SO₂—NR₅R₆, or
a group (((C₁-C₆)alkyl)₃)silylethynyl;
R₅ and R₆, which may be identical or different, represent a hydrogen atom or a group (C₁-C₆)alkyl;
Ra and Rb represent, independently of each other, a hydrogen atom or a group (C₁-C₆)alkyl;
Rc represents a hydrogen atom and Rd represents a hydrogen atom or a group (C₁-C₆)alkyl, the said alkyl being optionally substituted with a group (C₁-C₆)alkoxy;
or an acid addition salt thereof.

6. The compound of formula (I) according to claim 1, wherein:
X represents a phenyl group;
R₁ represents a hydrogen atom, a chlorine atom or a methyl or ethoxy group;
R₃ and R₄ represent a hydrogen atom;
R₂ represents a group NH₂, CH═NOH, NHiPr, nitro, CH═NOMe, NHMe, N═CHNMe₂, NHEt, NHCH₂CH₂OMe, SMe, SOMe, SO₂Me, SO₂NH₂, SO₂NHMe, SO₂NMe₂, or C≡CSiMe₃,
or an acid addition salt thereof.

7. The compound according to claim 1, selected from the group consisting of:
6-[(E)-(Hydroxyimino)methyl]-N-phenylimidazo[1,2-a]pyridine-2-carboxamide;
6-(Isopropylamino)-N-phenylimidazo[1,2-a]pyridine-2-carboxamide and its hydrochloride (1:1);
6-Nitro-N-phenylimidazo[1,2-a]pyridine-2-carboxamide;
6-Amino-N-phenylimidazo[1,2-a]pyridine-2-carboxamide;
6-Amino-5-chloro-N-phenylimidazo[1,2-a]pyridine-2-carboxamide;
6-Amino-5-ethoxy-N-phenylimidazo[1,2-a]pyridine-2-carboxamide;
6-{[(1E)-(Dimethylamino)methylene]amino}-N-phenylimidazo[1,2-a]pyridine-2-carboxamide;
6-[(E)-(Methoxyimino)methyl]-N-phenylimidazo[1,2-a]pyridine-2-carboxamide;
6-(Methylamino)-N-phenylimidazo[1,2-a]pyridine-2-carboxamide;
6-(Ethylamino)-N-phenylimidazo[1,2-a]pyridine-2-carboxamide;
6-(2-Methoxyethylamino)-N-phenylimidazo[1,2-a]pyridine-2-carboxamide;
5-Methyl-6-nitro-N-phenylimidazo[1,2-a]pyridine-2-carboxamide;
6-Amino-5-methyl-N-phenylimidazo[1,2-a]pyridine-2-carboxamide;
6-(Methylthio)-N-phenylimidazo[1,2-a]pyridine-2-carboxamide and its hydrobromide (1:1);
6-[(RS)-Methylsulfinyl]-N-phenylimidazo[1,2-a]pyridine-2-carboxamide and its hydrobromide (1:1);
6-(Methylsulfonyl)-N-phenylimidazo[1,2-a]pyridine-2-carboxamide and its hydrobromide (1:1);
6-(Aminosulfonyl)-N-phenylimidazo[1,2-a]pyridine-2-carboxamide and its hydrobromide (1:1);
6-[(Methylamino)sulfonyl]-N-phenylimidazo[1,2-a]pyridine-2-carboxamide and its hydrobromide (1:1);
6-[(Dimethylamino)sulfonyl]-N-phenylimidazo[1,2-a]pyridine-2-carboxamide; and
6-(Trimethylsilylethynyl)-N-phenylimidazo[1,2-a]pyridine-2-carboxamide and its hydrobromide (1:1);
or an addition salt thereof with a pharmaceutically acceptable acid.

8. A pharmaceutical composition comprising a compound of formula (I) according to claim 1, or a pharmaceutically acceptable salt thereof, and also at least one pharmaceutically acceptable excipient.

9. A pharmaceutical composition comprising a compound of formula (I) according to claim 7, or a pharmaceutically acceptable salt thereof, and also at least one pharmaceutically acceptable excipient.

10. A method of treating or preventing neurodegenerative diseases comprising administering to a patient an effective amount of a compound of formula (I) according to claim 1, or a pharmaceutically acceptable acid addition salt thereof.

11. A method of treating or preventing cerebral trauma or epilepsy comprising administering to a patient an effective amount of a compound of formula (I) according to claim 1, or a pharmaceutically acceptable acid addition salt thereof.

12. A method of treating or preventing psychiatric diseases comprising administering to a patient an effective amount of a compound of formula (I) according to claim 1, or a pharmaceutically acceptable acid addition salt thereof.

13. A method of treating or preventing inflammatory diseases comprising administering to a patient an effective amount of a compound of formula (I) according to claim 1, or a pharmaceutically acceptable acid addition salt thereof.

14. A method of treating or preventing osteoporosis comprising administering to a patient an effective amount of a compound of formula (I) according to claim 1, or a pharmaceutically acceptable acid addition salt thereof.

15. A method of treating or preventing cancers comprising administering to a patient an effective amount of a compound of formula (I) according to claim 1, or a pharmaceutically acceptable acid addition salt thereof.

16. A method of treating or preventing Parkinson's disease, Alzheimer's disease, tauopathies or multiple sclerosis comprising administering to a patient an effective amount of a compound of formula (I) according to claim 1, or a pharmaceutically acceptable acid addition salt thereof.

17. A method of treating or preventing preventing schizophrenia, depression, substance dependency or attention-deficit hyperactivity disorder comprising administering to a patient an effective amount of a compound of formula (I) according to claim 1, or a pharmaceutically acceptable acid addition salt thereof.

18. A compound selected from the group consisting of:
5-Ethylaminopyridine-2-amine; and
5-(2-Methoxyethylamino)pyridine-2-amine.
